(19) United States
(12) Patent Application Publication
(10) Pub. No.: US 2025/0161351 A1
(43) Pub. Date: May 22, 2025

(54) CANCER TREATMENT BY ARSENIC TRIOXIDE COMBINATION THERAPY

(71) Applicants: Deutsches Krebsforschungszentrum Stiftung des öffentlichen Rechts, Heidelberg (DE); HI-STEM GGMBH, Heidelberg (DE)

(72) Inventors: Andreas TRUMPP, Heidelberg (DE); Andreas NARR, Heidelberg (DE); Hamed ALBORZI NIA, Heidelberg (DE)

(73) Assignees: Deutsches Krebsforschungszentrum Stiftung des öffentlichen Rechts, Heidelberg (DE); HI-STEM GGMBH, Heidelberg (DE)

(21) Appl. No.: 18/872,264

(22) PCT Filed: Jun. 9, 2023
(86) PCT No.: PCT/EP2023/065436

§ 371 Date: Dec. 5, 2024

(30) Foreign Application Priority Data
Jun. 10, 2022 (EP) 22178340.0

(51) Int. Cl. A61K 33/36; A61K 31/198; A61K 31/501; A61K 31/635; A61K 31/706; A61P 35/02

(52) U.S. Cl. CPC A61K 33/36; A61K 31/198; A61K 31/501; A61K 31/635; A61K 31/706; A61P 35/02

(57) ABSTRACT

The present invention relates to an inhibitor of a glutathione peroxidase 4 (GPX4) for use in treating and/or preventing cancer in a subject in combination with (i) a Bcl-2 inhibitor and a hypomethylating agent, or (ii) a glutaminolysis inhibitor and a transsulfuration inhibitor, and to combined preparations, kit, methods, and uses related thereto.

The present invention relates to an inhibitor of a glutathione peroxidase 4 (GPX4) for use in treating and/or preventing cancer in a subject in combination with (i) a Bcl-2 inhibitor and a hypomethylating agent, or (ii) a glutaminolysis inhibitor and a transsulfuration inhibitor, and to combined preparations, kit, methods, and uses related thereto.

Acute myeloid leukemia (AML) remains a cancer with dismal prognosis, particularly in elderly or frail patients ineligible for high dose chemotherapy, as well as in patients with high risk disease. The survival of AML cells is dependent on the expression of anti-apoptotic factors such as Bcl-2. In recent years, Venetoclax, a potent Bcl-2 inhibitor (Konopleva et al., 2016), in combination with hypomethylating agents (HMAs) has replaced HMAs alone as standard of care treatment for AML patients unsuitable for intensive induction chemotherapy (DiNardo et al., 2020a). Moreover, Venetoclax has recently been added successfully to various high dose induction protocols, providing further evidence of its effectiveness in AML treatment beyond HMA combinations (DiNardo et al., 2021; Garcia et al., 2021). HMAs in combination with Venetoclax are also currently being evaluated as first line treatment for adult AML patients eligible for intensive induction chemotherapy such as cytarabine and daunorubicin.

Arsenic trioxide has been known mostly for its toxic and carcinogenic properties; more recently, however, the compound was proposed for treatment of cancer, e.g. in acute promyelocytic leukemia (Chen et al., 2001) and neuroblastoma (Feng et al. 2022). In neuroblastoma cells, arsenic trioxide was found to potentially induce ferroptosis and, thereby, inhibition of proliferation, which may be due to decreasing activity of glutathione peroxidase 4 (Feng et al. 2022). Also, combination of arsenic trioxide with the glutaminase inhibitor CB-839 was found to enhance the inhibiting effect (Gregory et al., 2019).

Nonetheless, there is a need for improved treatments of cancer, in particular AML and relapses thereof. The technical problem underlying the present invention may be seen as the provision of means and methods for complying with the aforementioned need. The technical problem is solved by the embodiments characterized in the claims and herein below.

In accordance, the present invention relates to an inhibitor of a glutathione peroxidase 4 (GPX4) for use in treating and/or preventing cancer in a subject in combination with (i) a Bcl-2 inhibitor and a hypomethylating agent, or (ii) a glutaminolysis inhibitor and a transsulfuration inhibitor.

Also, the present invention relates to a combination of a Bcl-2 inhibitor and a hypomethylating agent, or a combination of a glutaminolysis inhibitor and a transsulfuration inhibitor, for use in treating and/or preventing cancer in a subject in combination with an inhibitor of a GPX4.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof; thus, e.g. identifying “a cell” relates to identifying at least one cell, preferably to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “polypeptide”, as used herein, refers to a molecule consisting of several, typically at least 20 amino acids that are covalently linked to each other by peptide bonds. Molecules consisting of less than 20 amino acids covalently linked by peptide bonds are usually considered to be “peptides”. Preferably, the polypeptide comprises of from 50 to 1000, more preferably of from 75 to 1000, still more preferably of from 100 to 500, most preferably of from 110 to 400 amino acids. Preferably, the polypeptide is comprised in a fusion polypeptide and/or a polypeptide complex.

The term “glutathione peroxidase 4”, also referred to as “GPX4” is understood by the skilled person. Glutathione peroxidases are a family of enzymes catalyzing the reduction of inorganic and/or organic peroxides with concomitant oxidation of glutathione. Preferably, the GPX4 is a mammalian GPX4, more preferably is a human GPX4, still more preferably a human GPX4, most preferably comprising an amino acid sequence as shown in Genbank Acc No. NP_002076.2 or one of its isoforms. Also preferably, the GPX4 is a polypeptide comprising an amino acid sequence as shown in Genbank Acc No. NP_002076.2, NP_001034936.1, NP_001034937.1, or NP_001354761.1, or an amino acid sequence at least 80%, preferably at least 90%, more preferably at least 95%, still more preferably at least 98%, most preferably at least 99% identical thereto. More preferably, the GPX4 is a polypeptide consisting of an amino acid sequence as shown in Genbank Acc No. NP_002076.2, NP_001034936.1, NP_001034937.1, or NP_001354761.1, or an amino acid sequence at least 80%, preferably at least 90%, more preferably at least 95%, still more preferably at least 98%, most preferably at least 99% identical thereto. Still more preferably, the GPX4 is a polypeptide consisting of an amino acid sequence as shown in Genbank Acc No. NP_002076.2, NP_001034936.1, NP 001034937.1, or NP_001354761.1.

As referred to herein, the term “inhibitor of GPX4” includes any and all chemical compounds causing the activity of a GPX4 as specified herein to decrease. As referred to herein, the term includes indirect and direct inhibitors of GPX4, wherein the term “indirect inhibitor of GPX4” is used to relate to a compound decreasing the amount of GPX4 in a cell, e.g. a shRNA causing degradation of an mRNA encoding GPX4; and the term “direct inhibitor of a GPX4” is used to relate to a compound binding to, i.e. directly interacting with, a GPX4, thereby decreasing its activity. As the skilled person will understand, direct inhibition may be caused by a variety of mechanisms, e.g. via allosteric inhibition, by competitive inhibition, or by covalent modification of the GPX4, preferably in its active center, more preferably by covalent modification of the selenocysteine residue in its active center. Inhibitors of GPX4 are known in the art, e.g. RAS-selective lethal 3 (RSL3, CAS No. 1219810-16-8), ML 162 (CAS No. 1035072-16-2), or ML 210 (CAS No. 1360705-96-9). In the work underlying the instant invention, it was surprisingly found that arsenic trioxide is a direct inhibitor of GPX4. Thus, the inhibitor of a GPX4 is preferably selected from the list consisting of arsenic trioxide, RAS-selective lethal 3 (RSL3, CAS No. 1219810-16-8), ML 162 (CAS No. 1035072-16-2), and ML 210 (CAS No. 1360705-96-9). More preferably, the inhibitor of GPX4 is arsenic trioxide. For the avoidance of doubt, it is noted that compounds decreasing the concentration of a substrate of GPX4 in a cell, e.g. glutaminolysis inhibitors and transsulfuration inhibitors as specified herein below, are not included as inhibitors of GPX4 as the term is used herein. Thus, the inhibitor of GPX4 is not a glutaminolysis inhibitor and transsulfuration inhibitor.

The term “arsenic trioxide” is understood by the skilled person. Preferably, the term relates to arsenic (III) oxide, As₂O₃, preferably with CAS No. 1327-53-3. Pharmaceutic preparations of arsenic trioxide are e.g. marketed under the name Trisenox®.

The term “Bcl-2 inhibitor” is, in principle, known in the art to relate to any compound inhibiting the activity of the Bcl-2 polypeptide involved in regulation of cellular apoptosis. The amino acid sequence of e.g. the alpha isoform of human Bcl-2 is obtainable under Genbank Acc. No. NP_000624.2. Bcl-2 inhibitors are known in the art, e.g. Venetoclax (ABT-199, 4-[4-[[2-(4-Chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)benzamide, CAS No. 1257044-40-8), oblimersen (CAS No. 190977-41-4), ABT-737 (Cas No. 852808-04-9), and navitoclax (ABT-263, CAS No. 923564-51-6). Preferably, the Bcl-2 inhibitor is Venetoclax.

The term “hypomethylating agent” is understood by the skilled person to relate to each and every chemical compound having the activity of causing DNA demethylation, i.e. the loss of methyl groups from nucleobases comprised within DNA. Thus, preferably, the hypomethylating agent of the present invention is a compound causing, when contacted to a cell at an effective concentration, the degree of methylation of the DNA comprised in said cell to decrease. In the context of the term “decrease of the degree of methylation”, the term “degree of methylation” relates to the ratio of the number of methylated nucleobases, preferably 5-Methylcytosine residues, within a specific DNA subsequence, to the total number of (i.e. methylated and unmethylated) nucleobases, preferably Cytosine residues, within said specific DNA subsequence; the term decrease, accordingly, relates to a decrease of the value of the aforesaid ratio. Preferably, the hypomethylating agent is an inhibitor of at least one DNA methyltransferase. DNA methyltransferases are well known in the art and, preferably, fall under one of EC classes 2.1.172, 2.1.1.113, or 2.1.1.37. More preferably, the hypomethylating agent is an inhibitor of at least one DNA (cytosine-5-)-methyltransferase (EC 2.1.1.37). Even more preferably, the hypomethylating agent is an inhibitor of a mammalian DNA methyltransferase (DNMT), most preferably of DNMT1. Assays for measuring the activity of a DNMT are known in the art and are commercially available, e.g. the DNMT Activity/Inhibition Assay marketed by ActiveMotif®.

The hypomethylating agent may be a non-covalent inhibitor of DNMT activity or, more preferably, a covalent inhibitor of DNMT activity. Preferably, the hypomethylating agent is selected from the list of non-covalent inhibitors of DNMT activity consisting of 1-hydrazinylphthalazine (Hydralazine), 4-amino-N-(2-diethylaminoethyl)benzamide (Procainamide); 2-(diethylamino)ethyl 4-aminobenzoate (Procaine); 2-(1H)-pyrimidinone riboside (Zebularine); 2-amino-4-([[(2S,3S,4R,5R)-5-(6-amino-2-R1-9H-purin-9-yl)-3,4-dihydroxytetrahydrofuran-2-yl]methyl]sulfanyl) butanoic acid with R1 being chloro-, fluoro-, iodo-, methoxy-, methyl- or methylsulfanyl-; S-Tubercidinylhomocysteine; S-(N-(2-biphenyl-4-ylethyl) adenosyl)-L-homocysteine; S-(N-(2-biphenyl-4-ylethyl)-2-chloroadenosyl)-L-homocysteine; S-(N-phenylpropyladenosyl)-L-homocysteine; 8-aza-S-adenosyl-L-homocysteine; S-(N-(3,5-dimethoxybenzyl) adenosyl)-L-homocysteine; S-(N-(pyridin-4-ylmethyl) adenosyl)-L-homocysteine; S-(N-phenylethyladenosyl)-L-homocysteine; S-nebularinehomocysteine; S-(N-(2-biphenyl-4-ylethyl)-2-chloroadenosyl)-L-homocysteine; 1-deaza-S-adenosyl-L-homocysteine; 3-deaza-S-adenosyl-L-homocysteine; S-(1-deazaadenosyl)-L-homocysteine; S-(N-benzyladenosyl)-L-homocysteine; S-(N-phenyladenosyl)-L-homocysteine; and 1,2-dihydropyrimidin-2-one-5-methylene-(methylsolfonium)-adenosyl. More preferably, the hypomethylating agent is a covalent inhibitor of at least one DNA methyltransferase. Thus, more preferably, the hypomethylating agent is selected from the list consisting of Azacytidine (4-Amino-1-β-D-ribofuranosyl-1,3,5-(1H)triazin-2-one, CAS No. 320-67-2), 5-Aza-2′-deoxycytidine, Arabinosyl-5-azacytidine, 5-6-Dihydro-5-azacytidine, 5-Fluoro-2′-deoxycytidine, and Epigallocatechin-3-gallate. More preferably, the hypomethylating agent is selected from the list consisting of Azacytidine, 5-Aza-2′-deoxycytidine, and Arabinosyl-5-azacytidine, most preferably, the hypomethylating agent is Azacytidine (4-Amino-1-β-D-ribofuranosyl-1,3,5-(1H)triazin-2-one, CAS No. 320-67-2).

The term “glutaminolysis” is understood by the skilled person to relate to the biochemical pathway converting glutamine via glutamate to alpha-ketoglutarate. In accordance, an “inhibitor of glutaminolysis” is a compound inhibiting at least one of the aforesaid conversions, causing a preferably significant decrease of glutamate and/or alpha-ketoglutarate concentration(s) in a cell. Preferably, the glutaminolysis inhibitor is a glutaminase inhibitor; corresponding inhibitors are known in the art, e.g. CB-839, Bis-2-(5-phenylacetamido-1,3,4-thiadiazol-2-yl)ethyl sulfide (BPTES), azaserine, acivicin, 6-diazo-5-oxo-L-norleucine (DON), thiazolidine-2,4-dione derivatives, or Compound 968, all as reviewed in Akins et al., 2018. More preferably, the glutaminolysis inhibitor is CB-839 (Telaglenastat, CAS No. 1439399-58-2).

The term “transsulfuration” is understood by the skilled person to relate to the biochemical pathway involving the interconversion of cysteine and homocysteine through the intermediate cystathionine. In accordance, a “transsulfuration inhibitor” is a compound inhibiting at least one of the aforesaid conversions causing a preferably significant decrease of cysteine concentration in a cell. Preferably, the transsulfuration inhibitor is an inhibitor of cystathionine gamma-synthase (EC 2.5.1.48). Appropriate inhibitors are known in the art. Preferably, the transsulfuration inhibitor is propargylglycine (2-aminopent-4-ynoic acid), more preferably DL-propargylglycine (CAS No. 50428-03-0), even more preferably L-propargylglycine (CAS No. 23235-01-0).

The term “cancer”, as used herein, relates to a disease of an animal, including man, characterized by uncontrolled growth by a group of body cells (“cancer cells”). This uncontrolled growth may be accompanied by intrusion into and destruction of surrounding tissue (infiltration) and possibly spread of cancer cells to other locations in the body (metastasis). Preferably, also included by the term cancer is a recurrence of a cancer (relapse). Thus, preferably, the cancer is a solid cancer, a metastasis, or a relapse thereof. More preferably, the cancer is a non-solid cancer, in particular a leukemia, in particular a relapse or an advanced stage leukemia. Preferably, the cancer is selected from the list consisting of acute myeloid leukemia (AML), acute lymphoblastic leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, fibrosarcoma, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sézary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenström macroglobulinemia, and wilms tumor. More preferably, the cancer is leukemia, breast cancer, pancreatic ductal adenocarcinoma, ovarian cancer, B-cell lymphoma, renal cell carcinoma, lung cancer, or glioblastoma. Still more preferably, the cancer is a leukemia, preferably acute myeloid leukemia. Preferably, the cancer is not acute promyelocytic leukemia.

The terms “treating” and “treatment” refer to an amelioration of a disease or disorder referred to herein or the symptoms accompanied therewith to a significant extent; as used herein, the term includes prevention of deterioration of a disease, disorder, or symptoms associated therewith. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population. Preferably, treating comprises inhibiting proliferation, more preferably killing, of cancer cells. Preferably, treating cancer is reducing tumor and/or cancer cell burden in a subject. As will be understood by the skilled person, effectiveness of treatment of e.g. cancer is dependent on a variety of factors including, e.g. cancer stage and cancer type. Also preferably, cancer treatment further comprises at least one of chemotherapy, immunotherapy, surgery, and radiotherapy. Preferably, treating comprises treating a relapse, in particular of leukemia, e.g. of AML. Also preferably, treating comprises treating an advanced stage cancer, in particular of leukemia, e.g. of AML; preferably, said advanced stage is one of stages M4 to M7 in the French-American-British classification system, more preferably is stage M5, M6, or M7.

The terms “preventing” and “prevention” refer to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification. In the context of cancer treatment, preventing in particular relates to preventing cancer development, preventing metastasis formation, and/or preventing relapse, preferably relates to preventing metastasis formation and/or preventing relapse.

Preferably, treating and/or preventing cancer as described herein comprises combined use of an inhibitor of GPX4, a Bcl-2 inhibitor and a hypomethylating agent; or comprises combined use of an inhibitor of GPX4, a glutaminolysis inhibitor, and a transsulfuration inhibitor. Thus, preferably, treating and/or preventing cancer as described herein may in particular comprise combined use of arsenic trioxide, Venetoclax, and Azacytidine; or may comprise combined use of arsenic trioxide, CB-839, and propargylglycine.

The terms ““chemotherapy”, “surgery”, and “radiotherapy” are understood by the skilled person. Appropriate standard treatment protocols are available in the art. The term “immunotherapy”, as used herein, relates to the treatment and/or prevention of disease, preferably cancer, by modulation of the immune response of a subject. Said modulation may be inducing, enhancing, or suppressing said immune response, e.g. by administration of at least one immune checkpoint modulator and/or cytokine. Preferably, the cytokine is an interferon, an interleukin, or a chemokine in such case. The immunotherapy may also comprise administration of T cells, e.g. CAR T cells and/or recombinant T cell receptor T cells, and/or at least one T cell engager, i.e. a molecule tethering a T cell to a target cell; corresponding T cell engagers, e.g. bispecific T cell engagers (BiTEs) such as bispecific antibodies, are known in the art.

The term “subject”, as referred to herein, relates to a vertebrate animal, preferably a mammal, in particular a livestock, companion, or laboratory animal. More preferably, subject is a human. Preferably, the subject has been diagnosed to suffer from cancer as specified herein above and/or was diagnosed to be at risk of developing a relapse and/or metastases. In accordance with the above, a “cell” preferably is a eukaryotic cell, more preferably a vertebrate cell, most preferably a mammalian cell, still more preferably a human cell. Preferably, the cell is a cancer cell. Thus, preferably, a cell preferably is a mammalian cancer cell, more preferably a human cancer cell.

Advantageously, it was found in the work underlying the present invention that the combination therapies described herein have advantageous properties compared to the corresponding single treatments and provide for a synergistic effect in cancer treatment.

The definitions made above apply mutatis mutandis to the following. Additional definitions and explanations made further below also apply for all embodiments described in this specification mutatis mutandis.

In view of the above, the present invention also relates
to a Bcl-2 inhibitor for use in treating and/or preventing cancer in a subject in combination with an inhibitor of a GPX4 and a hypomethylating agent;
to a. hypomethylating agent for use in treating and/or preventing cancer in a subject in combination with a Bcl-2 inhibitor and an inhibitor of a GPX4;
to a glutaminolysis inhibitor for use in treating and/or preventing cancer in a subject in combination with an inhibitor of a GPX4 and a transsulfuration inhibitor; as well as to a
to a transsulfuration inhibitor for use in treating and/or preventing cancer in a subject in combination with a glutaminolysis inhibitor and an inhibitor of a GPX4.

The present invention further relates to a combined preparation comprising (a) an inhibitor of a GPX4 and (b) (i) a Bcl-2 inhibitor and a hypomethylating agent, or (ii) a glutaminolysis inhibitor and a transsulfuration inhibitor.

The term “combined preparation”, as referred to herein, relates to a preparation comprising the pharmaceutically active compounds as specified in one preparation. Preferably, the combined preparation is comprised in a container, i.e. preferably, said container comprises all pharmaceutically active compounds of the present invention. As will be understood by the skilled person, the term “formulation” relates to a, preferably pharmaceutically acceptable, mixture of compounds, comprising or consisting of the pharmaceutically active compounds as specified. Preferably, the combined preparation comprises an inhibitor of GPX4, a Bcl-2 inhibitor and a hypomethylating agent; or comprises an inhibitor of GPX4, a glutaminolysis inhibitor, and a transsulfuration inhibitor. Thus, preferably, the combined preparation comprises arsenic trioxide, Venetoclax, and Azacytidine; or comprises arsenic trioxide, CB-839, and propargylglycine. Preferably, said compounds are comprised in a single solid pharmaceutical form, e.g. a tablet; more preferably, the compounds are comprised in a liquid formulation; said liquid formulation, preferably, is for injection.

Preferably, the combined preparation is a pharmaceutical composition, said pharmaceutical composition preferably further comprising a pharmaceutically acceptable carrier. The terms “medicament” and “pharmaceutical composition” are used essentially interchangeably herein and are, in principle, known to the skilled person. As referred to herein, the terms relate to any composition of matter comprising the specified active agents as pharmaceutically active compounds and, optionally, one or more excipient. The pharmaceutically active compounds can be present in liquid or dry, e.g. lyophilized, form. It will be appreciated that the form and character of the pharmaceutical acceptable excipient, e.g. carrier or diluent, is dictated by the amount of active ingredient with which it is to be combined, the route of administration, and other well-known variables. The excipient(s) must be acceptable in the sense of being compatible with the other ingredients of the formulation and being not deleterious to the recipient thereof. The excipient employed may include a solid, a gel, or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, physiological saline, Ringer's solutions, dextrose solution, and Hank's solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania. The excipient(s) is/are selected so as not to affect the biological activity of the combination. The excipient may, however, also be selected to improve uptake of the active agent into a cell, in particular a cancer cell.

The medicament is, preferably, administered by a route as specified herein above. A therapeutically effective dose refers to an amount of the active compound which prevents, ameliorates or cures the symptoms accompanying a disease or condition referred to in this specification. Therapeutic efficacy and toxicity of a drug can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and by clinical factors. As is well known in the medical arts, dosages for any one patient depends upon many factors, including the patient's size, age, the particular formulation of the medicament to be administered, sex, time and route of administration, general health, and other drugs being administered concurrently. The medicament referred to herein is, preferably, administered at least once, e.g. as a bolus. However, the medicament may be administered more than one time and, preferably, at least twice, e.g. permanently or periodically after defined time windows. Progress can be monitored by periodic assessment. Dosage recommendations may be indicated in the prescriber or user instructions in order to anticipate dose adjustments depending on the considered recipient. As will be understood by the skilled person, appropriate doses for the pharmaceutically active compounds described herein are known in the art for single use of said compounds. Thus, a dose may be in particular such a dose known in the art. However, in view of the synergistic effects described herein, it is also envisaged that the doses known in the art are reduced, preferably by a factor of at least 2, more preferably a factor of at least 3, even more preferably a factor of at least 5, most preferably a factor of at least 10. It will also be understood by the skilled person that in a combined treatment as described herein, also only the dose of only one or only two pharmaceutically active compounds may be reduced compared to the known dose, while the dose of the remaining pharmaceutically active compound(s) may be kept unmodified; in such case, preferably, the dose of the pharmaceutically active compound(s) causing the most severe adverse reactions is reduced.

The medicament according to the present invention may comprise further active agents in addition to the aforementioned active agent(s). Preferably, the pharmaceutically active compounds according to the invention are to be applied together with at least one further drug and, thus, may be formulated together with this at least one further drug as a medicament. More preferably, in case of cancer treatment, said at least one further active agent is a chemotherapeutic agent or an immunotherapeutic agent, such as a T cell or an immune checkpoint modulator. Also, it is to be understood that the formulation of a pharmaceutical composition preferably takes place under GMP standardized conditions or the like in order to ensure quality, pharmaceutical safety, and effectiveness of the medicament.

The present invention also relates to the combined preparation as specified herein above for use in medicine; and for use in treating and/or preventing cancer.

The present invention further relates to a kit comprising (a) an inhibitor of a GPX4 and (b) (i) a Bcl-2 inhibitor and a hypomethylating agent, or (ii) a glutaminolysis inhibitor and a transsulfuration inhibitor, preferably comprised in a housing.

The term “kit”, as used herein, refers to a collection of the aforementioned compounds, means or reagents which may or may not be packaged together. The components of the kit may be comprised by separate vials (i.e. as a kit of separate parts) or provided in a single vial, e.g. as a composition as specified herein above. The housing of the kit preferably allows translocation of the compounds of the kit, in particular common translocation; thus, the housing may in particular be a transportable container comprising all specified components. Moreover, it is to be understood that the kit of the present invention may be used for practicing the methods referred to herein. It is preferably envisaged that all components are provided in a ready-to-use manner for practicing the methods referred to above. Further, the kit preferably contains instructions for carrying out said methods. The instructions can be provided by a user's manual on paper or in electronic form. Preferably, the kit is adapted for use in a method of the present invention, more preferably is adapted to comprise all reagents required to perform said method or methods. Also preferably, the kit comprises the compounds as specified in single doses, i.e. comprises the compounds in amounts corresponding to a single dose to be administered to a subject. As specified herein above, such doses may be decreased compared to the doses known in the art.

Preferably, the compounds in the kit are for separate or for combined administration. “Separate administration”, as used herein, relates to an administration wherein at least two of the pharmaceutically active compounds of the present invention are administered via different routes and/or at different parts of the body of a subject. E.g. one compound may be administered by enteral administration (e.g. orally), whereas a second compound is administered by parenteral administration (e.g. intravenously). Preferably, in such case the kit comprises at least two physically separated preparations for separate administration, wherein each preparation contains at least one pharmaceutically active compound; said alternative is preferred e.g. in cases where the pharmaceutically active compounds of the combined preparation have to be administered by different routes, e.g. parenterally and orally, due to their chemical or physiological properties. Conversely, “combined administration” relates to an administration wherein the pharmaceutically active compounds of the present invention are administered via the same route, e.g. orally or, preferably, intravenously. Thus, in such case, the kit may comprise a preparation comprising at least two, preferably all, pharmaceutically active compounds in a single preparation. Thus, the kit may preferably comprise a combined preparation as specified herein above.

Also preferably, the compounds in the kit are for simultaneous or for sequential administration. “Simultaneous administration”, as used herein, relates to an administration wherein the pharmaceutically active compounds of as specified are administered at the same time, i.e., preferably, administration of the pharmaceutically active compounds starts within a time interval of less than 15 minutes, more preferably, within a time interval of less than 5 minutes. Most preferably, administration of the pharmaceutically active compounds starts at the same time, e.g. by swallowing a tablet comprising the pharmaceutically active compounds, or by swallowing a tablet comprising one of the pharmaceutically active compounds and simultaneous injection of the second compound, or by applying an intravenous injection of a solution comprising one pharmaceutically active compound and injecting second compound in different part of the body. Conversely, “sequential administration”, as used herein, relates to an administration causing plasma concentrations of the pharmaceutically active compounds in a subject enabling the synergistic effect of the present invention, but which, preferably, is not a simultaneous administration as specified herein above. Preferably, sequential administration is an administration wherein administration of the pharmaceutically active compounds, preferably all pharmaceutically active compounds, starts within a time interval of 1 or 2 days, more preferably within a time interval of 12 hours, still more preferably within a time interval of 4 hours, even more preferably within a time interval of one hour, most preferably within a time interval of 5 minutes.

The present invention also relates to a method of treating and/or preventing cancer in a subject, said method comprising administering to said subject (a) an inhibitor of a GPX4 and (b) (i) a Bcl-2 inhibitor and a hypomethylating agent, or (ii) a glutaminolysis inhibitor and a transsulfuration inhibitor, thereby treating and/or preventing cancer.

The method of treating and/or preventing cancer of the present invention, preferably, is an in vivo method. Moreover, it may comprise steps in addition to those explicitly mentioned above. For example, further steps may relate, e.g., to diagnosing cancer before treating, and/or further treatments as specified herein above, which may be administered preceding, concomitant to, or following the specified treatment. Moreover, one or more of the treatment steps may be assisted or performed by automated equipment.

The present invention also relates to a method of inhibiting cancer cells, said method comprising contacting said cells with (a) an inhibitor of a GPX4 and (b) (i) a Bcl-2 inhibitor and a hypomethylating agent, or (ii) a glutaminolysis inhibitor and a transsulfuration inhibitor, thereby inhibiting cancer cells.

The method of inhibiting cancer cells of the present invention, preferably, is an in vitro method. It may, however, also be an in vivo method, e.g. as part of a method of treating cancer, preferably as specified herein above. Moreover, the method may comprise steps in addition to those explicitly mentioned above. For example, further steps may relate, e.g., to providing and cultivating cancer cells, and/or further treatments, which may be administered preceding, concomitant to, or following the specified treatment. Moreover, one or more of the treatment steps may be assisted or performed by automated equipment.

The term “inhibiting cancer cells”, as used herein, relates to inhibiting proliferation of cancer cells; thus, inhibition may be established by counting the number of cancer cells obtained after contacting and comparing said number to the number of cancer cells in an untreated control, preferably as specified herein in the Examples. Preferably inhibiting cancer cells comprises killing at least a fraction of cancer cells. Also preferably, inhibiting cancer cells comprises inducing ferroptosis in at least a fraction of cancer cells.

The term “ferroptosis” is known to the skilled person to relate to a type of programmed cell death dependent on iron and characterized by the accumulation of lipid peroxides in a cell.

The present invention also relates to a use, preferably in vitro use, of (a) an inhibitor of a GPX4 and (b) (i) a Bcl-2 inhibitor and a hypomethylating agent, or (ii) a glutaminolysis inhibitor and a transsulfuration inhibitor for inhibiting cancer cells, preferably for inducing ferroptosis in said cancer cells.

The present invention further relates to a use of a combined preparation of the present invention and/or a kit of the present invention in the manufacture of a medicament for treating and/or preventing cancer.

In view of the above, the following embodiments are particularly envisaged:

Embodiment 1: An inhibitor of a glutathione peroxidase 4 (GPX4) for use in treating and/or preventing cancer in a subject in combination with (i) a Bcl-2 inhibitor and a hypomethylating agent, or (ii) a glutaminolysis inhibitor and a transsulfuration inhibitor.

Embodiment 2: The inhibitor of GPX4 for use of embodiment 1, wherein said inhibitor of GPX4 is a direct inhibitor of GPX4.

Embodiment 3: The inhibitor of GPX4 for use of embodiment 1 or 2, wherein said inhibitor of GPX4 is arsenic trioxide (CAS No. 1327-53-3), RAS-selective lethal 3 (RSL3, CAS No. 1219810-16-8), ML 162 (CAS No. 1035072-16-2), or ML 210 (CAS No. 1360705-96-9).

Embodiment 4: The inhibitor of GPX4 for use of any one of embodiments 1 to 3, wherein said inhibitor of GPX4 is arsenic trioxide.

Embodiment 5: The inhibitor of GPX4 for use of any one of embodiments 1 to 4, wherein said inhibitor of GPX4 is used in combination with a Bcl-2 inhibitor and a hypomethylating agent.

Embodiment 6: The inhibitor of GPX4 for use of any one of embodiments 1 to 5, wherein said Bcl-2 inhibitor is Venetoclax (4-[4-[[2-(4-Chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)benzamide, CAS No. 1257044-40-8).

Embodiment 7: The inhibitor of GPX4 for use of any one of embodiments 1 to 6, wherein said hypomethylating agent is Azacytidine (4-Amino-1-β-D-ribofuranosyl-1,3,5-(1H)triazin-2-one, CAS No. 320-67-2).

Embodiment 8: The inhibitor of GPX4 for use of any one of embodiments 1 to 4, wherein said inhibitor of GPX4 is used in combination with a glutaminolysis inhibitor and a transsulfuration inhibitor.

Embodiment 9: The inhibitor of GPX4 for use of any one of embodiments 1 to 4 and 8, wherein said glutaminolysis inhibitor is a glutaminase inhibitor, preferably is CB-839 (Telaglenastat, CAS No. 1439399-58-2).

Embodiment 10: The inhibitor of GPX4 for use of any one of embodiments 1 to 4 and 8 or 9, wherein said transsulfuration inhibitor is an inhibitor of cystathionine gamma-synthase (EC 2.5.1.48), more preferably is propargylglycine (2-aminopent-4-ynoic acid), more preferably DL-propargylglycine (CAS No. 50428-03-0), even more preferably L-propargylglycine (CAS No. 23235-01-0).

Embodiment 11: The inhibitor of GPX4 for use of any one of embodiments 1 to 10, wherein said GPX4 is a mammalian GPX4, preferably is a human GPX4, more preferably a human GPX4 comprising an amino acid sequence as shown in Genbank Acc No. NP_002076.2 or one of its isoforms.

Embodiment 12: The inhibitor of GPX4 for use of any one of embodiments 1 to 11, wherein said cancer is leukemia, breast cancer, pancreatic ductal adenocarcinoma, ovarian cancer, B-cell lymphoma, renal cell carcinoma, lung cancer, or glioblastoma.

Embodiment 13: The inhibitor of GPX4 for use of embodiment 12, wherein said leukemia is acute myeloid leukemia.

Embodiment 14: The inhibitor of GPX4 for use of any one of embodiments 1 to 13, wherein said cancer is not acute promyelocytic leukemia.

Embodiment 15: The inhibitor of GPX4 for use of any one of embodiments 1 to 14, wherein said cancer is a relapse or advanced stage cancer.

Embodiment 16: The inhibitor of GPX4 for use of any one of embodiments 1 to 15, wherein said subject is a mammal, preferably a human.

Embodiment 17: A combination of a Bcl-2 inhibitor and a hypomethylating agent, or a combination of a glutaminolysis inhibitor and a transsulfuration inhibitor for use in treating and/or preventing cancer in a subject in combination with an inhibitor of a GPX4.

Embodiment 18: A combined preparation comprising (a) an inhibitor of a GPX4 and (b) (i) a Bcl-2 inhibitor and a hypomethylating agent, or (ii) a glutaminolysis inhibitor and a transsulfuration inhibitor.

Embodiment 19: The combined preparation of embodiment 18, wherein said combined preparation is a pharmaceutical combined preparation.

Embodiment 20: The combined preparation of embodiment 18 or 19, wherein said combined preparation comprises an inhibitor of a GPX4, a Bcl-2 inhibitor, and a hypomethylating agent.

Embodiment 21: The combined preparation of any one of embodiments 18 to 19, wherein said combined preparation comprises an inhibitor of a GPX4, a glutaminolysis inhibitor, and a transsulfuration inhibitor.

Embodiment 22: The combined preparation of any one of embodiments 18 to 21 for use in medicine.

Embodiment 23: The combined preparation of any one of embodiments 18 to 21 for use in treating and/or preventing cancer.

Embodiment 24: A kit comprising (a) an inhibitor of a GPX4 and (b) (i) a Bcl-2 inhibitor and a hypomethylating agent, or (ii) a glutaminolysis inhibitor and a transsulfuration inhibitor, preferably comprised in a housing.

Embodiment 25: The kit of embodiment 24, wherein the components comprised in the kit are for simultaneous, separate or sequential use.

Embodiment 26: A method of treating and/or preventing cancer in a subject, said method comprising administering to said subject (a) an inhibitor of a GPX4 and (b) (i) a Bcl-2 inhibitor and a hypomethylating agent, or (ii) a glutaminolysis inhibitor and a transsulfuration inhibitor, thereby treating and/or preventing cancer.

Embodiment 27: A method, preferably an in vitro method, of inhibiting cancer cells, said method comprising contacting said cells with (a) an inhibitor of a GPX4 and (b) (i) a Bcl-2 inhibitor and a hypomethylating agent, or (ii) a glutaminolysis inhibitor and a transsulfuration inhibitor, thereby inhibiting cancer cells.

Embodiment 28: Use, preferably in vitro use, of (a) an inhibitor of a GPX4 and (b) (i) a Bcl-2 inhibitor and a hypomethylating agent, or (ii) a glutaminolysis inhibitor and a transsulfuration inhibitor for inhibiting cancer cells, preferably for inducing ferroptosis in said cancer cells.

Embodiment 29: Use of a combined preparation according to any one of embodiments 18 to 21 and/or of the kit according to embodiment 24 or 25 in the manufacture of a medicament for treating and/or preventing cancer.

All references cited in this specification are herewith incorporated by reference with respect to their entire disclosure content and the disclosure content specifically mentioned in this specification.

### FIGURE LEGENDS

FIG. 1: Synergy Score values calculated as described in Example 4 for a variety of compound combinations; (A) Nomo-1 cells; (B) HL-60 cells.

FIG. 2: Synergy Score values calculated as described in Example 5 for a variety of compound combinations.

The following Examples shall merely illustrate the invention. They shall not be construed, whatsoever, to limit the scope of the invention.

### Example 1: Cell Viability Measurement AML Cell Lines

In order to assess viability of cells, 50,000 cells/well were plated into a flat-bottom 96-well plate in 150 μl medium per condition. Cells were treated with the stated drug concentrations for 72 h. After 72 h of treatment 15 μl/well of Cell Titer Blue Reagent was added and incubated at 37° C. in the cell incubator for 3 h. Then, fluorescence was measured using a SpectraMax Platereader (Excitation: 560 nm/Emission: 590 nm). Normalization of measured values was done to vehicle treated replicates. Cell lines were cultured in 10% FCS supplemented RPMI1640 or DMEM GlutaMax Medium.

### Example 2: Cell Viability Measurement Primary AML Samples

In order to assess viability of primary AML cells, 20,000 cells/well were plated into a flat-bottom 96-well plate in 150 μl medium per condition. Cells were treated with the stated drug concentrations for 72 h. After 72 h of treatment, cell viability was assessed using the Cell-Titer-Glo Luminescence Kit according to the manufacturer's protocol for suspension cells. Normalization of measured values was done to vehicle treated replicates. Primary AML samples were cultured in X-VIVO Medium supplemented with 15% BIT, 100 ng/ml SCF, 50 ng/ml Flt3L, 20 ng/ml IL-3, 20 ng/ml GCSF.

### Example 3: Combined treatment of AML cells with arsenic trioxide, Venetoclax, and Azacytidine

AML cancer cell lines Nomo-1 and HL60 were treated with the concentrations of arsenic trioxide, Venetoclax, and Azacytidine indicated in Tables 1 and 2, and cell viability was determined as described in Example 1.

TABLE 1
Relative cell viability compared to control in % after
the indicated treatment, Nomo-1 cells; ATO: arsenic
trioxide; values are means of independent duplicates.

| Venetoclax [nM] + | | | |
|---|---|---|---|
| 1.5 μM | ATO [μM] | | |
| Azacytidine | 0 | 0.5 | 1 |
| 0 | 100.0 | 80.8 | 73.1 |
| 7.8 | 103.2 | 44.0 | 58.1 |
| 15.6 | 102.1 | 34.8 | 44.1 |
| 31.3 | 95.7 | 38.2 | 38.2 |
| 62.5 | 94.6 | 28.1 | 30.8 |
| 125 | 71.6 | 15.3 | 20.4 |
| 250 | 61.0 | 9.1 | 14.3 |
| 500 | 46.7 | 9.9 | 12.6 |

TABLE 2
Relative cell viability compared to control in % after the indicated
treatment, HL-60 cells; values are means of independent duplicates.

| Venetoclax [nM] + | | | |
|---|---|---|---|
| 1.5 μM | ATO [μM] | | |
| Azacytidine | 0 | 0.5 | 1 |
| 0 | 100.0 | 113.4 | 106.3 |
| 7.8 | 75.1 | 88.2 | 48.7 |
| 15.6 | 69.9 | 75.7 | 24.8 |
| 31.3 | 69.6 | 66.8 | 22.7 |
| 62.5 | 74.9 | 49.3 | 19.6 |
| 125 | 68.7 | 25.5 | 19.8 |
| 250 | 45.6 | 21.4 | 20.4 |
| 500 | 30.1 | 21.4 | 20.4 |

### Example 4: Combined Treatment of AML Cells with Arsenic Trioxide, CB-839, and Propargylglycine

AML cancer cell lines Nomo-1 and HL60 and patient-derived primary AML cells HD-23 were treated with the concentrations of arsenic trioxide, CB-839, and propargylglycine indicated in Tables 3 to 5, and cell viability was determined as indicated in Examples 1 or 2, respectively.

TABLE 3
Relative cell viability compared to control in % after the indicated
treatment, Nomo-1 cells; ATO: arsenic trioxide, PPG: propargylglycine;
values are means of independent triplicates.

CB-839 [nM]

| ATO [μM] | PPG [mM] | 0 | 250 | 500 | 1000 |
|---|---|---|---|---|---|
| 0 | 0 | 100.0 | 92.5 | 96.1 | 98.1 |
| 0 | 0.25 | 97.9 | 91.1 | 91.0 | 93.8 |
| 0 | 0.5 | 96.6 | 93.4 | 92.6 | 97.1 |
| 0 | 1 | 95.2 | 91.0 | 92.6 | 93.7 |
| 0.5 | 0 | 107.9 | 98.4 | 99.7 | 100.4 |
| 0.5 | 0.25 | 108.0 | 100.8 | 99.7 | 100.2 |
| 0.5 | 0.5 | 106.1 | 102.4 | 101.5 | 97.9 |
| 0.5 | 1 | 102.9 | 98.4 | 94.3 | 96.7 |
| 1 | 0 | 96.3 | 88.5 | 88.5 | 87.4 |
| 1 | 0.25 | 93.2 | 80.0 | 81.0 | 81.6 |
| 1 | 0.5 | 95.4 | 74.5 | 75.0 | 73.8 |
| 1 | 1 | 90.7 | 59.2 | 58.9 | 59.6 |
| 2 | 0 | 63.9 | 24.9 | 25.5 | 25.0 |
| 2 | 0.25 | 57.0 | 20.8 | 21.0 | 20.0 |
| 2 | 0.5 | 52.0 | 18.5 | 18.1 | 18.2 |
| 2 | 1 | 38.6 | 15.7 | 14.6 | 14.3 |

TABLE 4
Relative cell viability compared to control in % after the indicated
treatment, HL-60 cells; ATO: arsenic trioxide, PPG: propargylglycine;
values are means of independent triplicates.

CB-839 [nM]

| ATO [μM] | PPG [mM] | 0 | 250 | 500 | 1000 |
|---|---|---|---|---|---|
| 0 | 0 | 100.0 | 94.5 | 95.1 | 92.1 |
| 0 | 0.25 | 98.7 | 91.0 | 91.5 | 91.5 |
| 0 | 0.5 | 98.9 | 88.5 | 92.0 | 92.4 |
| 0 | 1 | 97.9 | 89.0 | 85.3 | 85.7 |
| 0.5 | 0 | 107.6 | 89.0 | 85.1 | 80.7 |
| 0.5 | 0.25 | 107.6 | 82.3 | 78.5 | 75.2 |
| 0.5 | 0.5 | 104.0 | 81.8 | 76.0 | 64.3 |
| 0.5 | 1 | 96.8 | 46.5 | 34.8 | 33.1 |
| 1 | 0 | 82.3 | 26.2 | 15.3 | 10.6 |
| 1 | 0.25 | 86.8 | 31.1 | 15.7 | 7.9 |
| 1 | 0.5 | 81.2 | 22.2 | 10.2 | 4.0 |
| 1 | 1 | 79.6 | 16.6 | 5.9 | 2.7 |

TABLE 5
Relative cell viability compared to control in % after the indicated
treatment, HD-23 cells; ATO: arsenic trioxide, PPG: propargylglycine;
values are means of independent duplicates.

CB-839 [nM]

| ATO [μM] | PPG [mM] | 0 | 1000 |
|---|---|---|---|
| 0 | 0 | 100.0 | 84.6 |
| 0 | 2 | 92.9 | 57.7 |
| 2 | 0 | 46.5 | 15.8 |
| 2 | 2 | 36.3 | 5.6 |

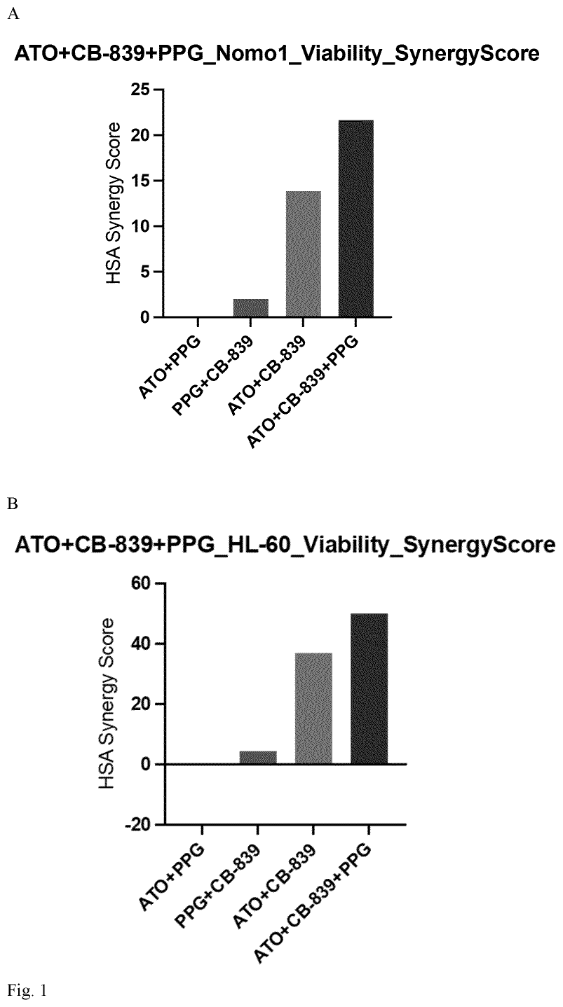

Further, from the above data for Nomo-1 and HL-60 cells, a Synergy Score for the interactions of the indicated compounds was calculated using the R-package synergyfinder; a Synergy Score of −10 to 10 is indicative of a probably additive interaction, while a Synergy Score higher than 10 is indicative of a probably synergistic interaction. Results are shown in FIG. 1. Combinations of arsenic trioxide and CB-839, and triple combinations of arsenic trioxide, propargylglycine, and CB-839 were found to have a synergistic effect in both cell lines.

### Example 5: Combined Treatment of Nomo-1 AML Cells with Arsenic Trioxide, Venetoclax, and Azacytidine

AML cancer cell line Nomo-1 was treated with the concentrations of arsenic trioxide, venetoclax, and azacytidine as indicated in Table 6, and cell viability was determined as indicated in Examples 1 or 2, respectively.

TABLE 6
Relative cell viability compared to control in % after
the indicated treatment, Nomo-1 cells; ATO: arsenic trioxide;
values are means of independent triplicates.

| | | |
|---|---|---|
| Venetoclax | Azacytidine | ATO [μM] |

| [nM] | [μM] | 0 | 0.5 | 1 | 2 |
|---|---|---|---|---|---|
| 0 | 0 | 100.0 | 95.6 | 70.3 | 27.5 |
| 15.625 | 0 | 93.8 | 86.8 | 61.6 | 16.7 |
| 31.25 | 0 | 90.4 | 82.1 | 55.8 | 16.6 |
| 62.5 | 0 | 85.4 | 65.7 | 38.2 | 7.3 |
| 125 | 0 | 79.8 | 53.0 | 32.3 | 5.4 |
| 250 | 0 | 77.4 | 50.7 | 29.1 | 3.9 |
| 500 | 0 | 63.6 | 41.5 | 20.9 | 2.5 |
| 1000 | 0 | 55.3 | 30.8 | 13.0 | 1.4 |
| 0 | 0.75 | 84.1 | 80.4 | 68.4 | 37.8 |
| 15.625 | 0.75 | 60.8 | 53.1 | 38.1 | 16.6 |
| 31.25 | 0.75 | 54.5 | 42.9 | 30.3 | 11.9 |
| 62.5 | 0.75 | 47.4 | 31.7 | 22.8 | 6.1 |
| 125 | 0.75 | 38.8 | 26.5 | 16.9 | 3.3 |
| 250 | 0.75 | 34.5 | 23.0 | 14.0 | 2.3 |
| 500 | 0.75 | 27.4 | 18.3 | 9.7 | 1.2 |
| 1000 | 0.75 | 19.3 | 11.0 | 4.8 | −0.2 |
| 0 | 1.5 | 67.1 | 70.8 | 64.0 | 40.7 |
| 15.625 | 1.5 | 44.6 | 36.7 | 30.7 | 12.7 |
| 31.25 | 1.5 | 38.6 | 31.9 | 23.8 | 9.1 |
| 62.5 | 1.5 | 34.4 | 27.3 | 20.3 | 5.4 |
| 125 | 1.5 | 28.7 | 22.0 | 15.1 | 3.2 |
| 250 | 1.5 | 23.7 | 17.0 | 11.4 | 2.1 |
| 500 | 1.5 | 20.0 | 13.6 | 7.8 | 0.7 |
| 1000 | 1.5 | 14.5 | 8.5 | 4.2 | 0.0 |

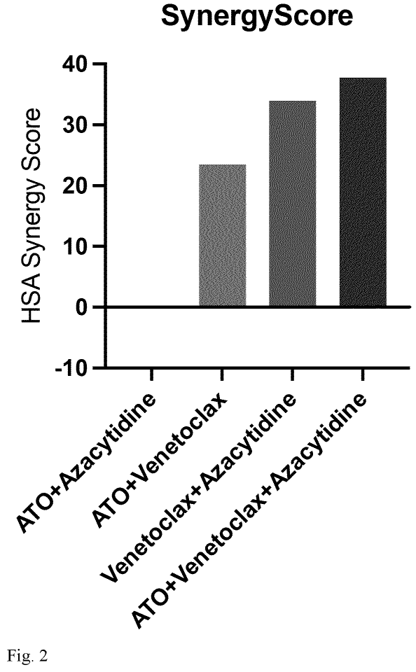

Further, from the above data, a Synergy Score for the interactions of the indicated compounds was calculated using the R-package synergyfinder, as indicated in Example 4. Results are shown in FIG. 2. Combinations of arsenic trioxide with venetoclax, combination of venetoclax with azacytidine, as well as the triple combination of arsenic trioxide, venetoclax, and azacytidine, were found to have a synergistic effect.

### CITED LITERATURE

Konopleva, M., Pollyea, D. A., Potluri, J., Chyla, B., Hogdal, L., Busman, T., McKeegan, E., Salem, A. H., Zhu, M., Ricker, J. L., et al. (2016). Efficacy and Biological Correlates of Response in a Phase II Study of Venetoclax Monotherapy in Patients with Acute Myelogenous Leukemia. Cancer Discov 6, 1106-1117
DiNardo, C. D., Jonas, B. A., Pullarkat, V., Thirman, M. J., Garcia, J. S., Wei, A. H., Konopleva, M., Dohner, H., Letai, A., Fenaux, P., et al. (2020a). Azacitidine and Venetoclax in Previously Untreated Acute Myeloid Leukemia. N Engl J Med 383, 617-629.
DiNardo, C. D., Lachowiez, C. A., Takahashi, K., Loghavi, S., Xiao, L., Kadia, T., Daver, N., Adeoti, M., Short, N. J., Sasaki, K., et al. (2021). Venetoclax Combined With FLAG-IDA Induction and Consolidation in Newly Diagnosed and Relapsed or Refractory Acute Myeloid Leukemia. J Clin Oncol 39, 2768-2778.;
Garcia, J. S., Kim, H. T., Murdock, H. M., Cutler, C. S., Brock, J., Gooptu, M., Ho, V. T., Koreth, J., Nikiforow, S., Romee, R., et al. (2021). Adding Venetoclax to fludarabine/busulfan RIC transplant for high-risk MDS and AML is feasible, safe, and active. Blood Adv 5, 5536-5545
Chen Z, Chen G Q, Shen Z X, Chen S J, Wang Z Y. Treatment of acute promyelocytic leukemia with arsenic compounds: in vitro and in vivo studies. Semin Hematol. 2001 January; 38 (1): 26-36. doi: 10.1053/shem.2001.20863. PMID: 11172537
Feng, C., Wu, Y., Chen, Y. et al. Arsenic trioxide increases apoptosis of SK-N-BE (2) cells partially by inducing GPX4-mediated ferroptosis. Mol Biol Rep (2022). doi.org/10.1007/s11033-022-07497-9
Gregory M A, Nemkov T, Park H J, Zaberezhnyy V, Gehrke S, Adane B, Jordan C T, Hansen K C, D'Alessandro A, DeGregori J. Targeting Glutamine Metabolism and Redox State for Leukemia Therapy. Clin Cancer Res. 2019 Jul. 1; 25 (13): 4079-4090. doi: 10.1158/1078-0432.CCR-18-3223
Akins N S, Nielson T C, Le H V. Inhibition of Glycolysis and Glutaminolysis: An Emerging Drug Discovery Approach to Combat Cancer. Curr Top Med Chem. 2018; 18 (6): 494-504. doi: 10.2174/1568026618666180523111351. PMID: 29788892; PMCID: PMC6110043

1-15. (canceled)

16. A method for inhibiting cancer cells, comprising contacting the cancer cells with an inhibitor of a glutathione peroxidase 4 (GPX4) and with (i) a Bcl-2 inhibitor and a hypomethylating agent, or (ii) a glutaminolysis inhibitor and a transsulfuration inhibitor.

17. The method of claim 16, wherein the inhibitor of GPX4 is arsenic trioxide (CAS No. 1327-53-3), RAS-selective lethal 3 (RSL3, CAS No. 1219810-16-8), ML 162 (CAS No. 1035072-16-2), or ML 210 (CAS No. 1360705-96-9).

18. The method of claim 16, wherein the inhibitor of GPX4 is arsenic trioxide.

19. The method of claim 16, wherein the inhibitor of GPX4 is used in combination with a Bcl-2 inhibitor and a hypomethylating agent.

20. The method of claim 16, wherein the Bcl-2 inhibitor is Venetoclax (4-[4-[[2-(4-Chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)benzamide, CAS No. 1257044-40-8).

21. The method of claim 16, wherein the hypomethylating agent is Azacytidine (4-Amino-1-β-D-ribofuranosyl-1,3,5-(1H)triazin-2-one, CAS No. 320-67-2).

22. The method of claim 16, wherein the inhibitor of GPX4 is used in combination with a glutaminolysis inhibitor and a transsulfuration inhibitor, preferably wherein the glutaminolysis inhibitor is a glutaminase inhibitor, more preferably is CB-839 (Telaglenastat, CAS No. 1439399-58-2); and/or preferably wherein the transsulfuration inhibitor is an inhibitor of cystathionine gamma-synthase (EC 2.5.1.48), more preferably is propargylglycine (2-aminopent-4-ynoic acid).

23. The method of claim 16, wherein the cancer is leukemia, breast cancer, pancreatic ductal adenocarcinoma, ovarian cancer, B-cell lymphoma, renal cell carcinoma, lung cancer, or glioblastoma, and/or wherein said cancer is a relapse or an advanced stage cancer.

24. The method of claim 23, wherein the leukemia is acute myeloid leukemia.

25. The method of claim 16, wherein the method is a method of treating and/or preventing cancer in a subject.

26. A combined preparation comprising (a) an inhibitor of a GPX4 and (b) (i) a Bcl-2 inhibitor and a hypomethylating agent, or (ii) a glutaminolysis inhibitor and a transsulfuration inhibitor.

27. A method of treating and/or preventing cancer in a subject, comprising contacting the subject with the combined preparation of claim 26.

28. A kit comprising (a) an inhibitor of a GPX4 and (b) (i) a Bcl-2 inhibitor and a hypomethylating agent, or (ii) a glutaminolysis inhibitor and a transsulfuration inhibitor, preferably comprised in a housing, preferably wherein the components comprised in the kit are for simultaneous, separate or sequential use.

29. The method of claim 16, wherein inhibition of the cancer cells comprises inducing ferroptosis in the cancer cells.
